(19) United States
(12) Patent Application Publication
(10) Pub. No.: US 2012/0310216 A1
(43) Pub. Date: Dec. 6, 2012

(54) INTEGRATED ORAL GASTRIC TUBE GUIDE

(75) Inventors: Vladimir Koltchine, Redding, CT (US); Eric Kitain, Redding, CT (US); Robert Koorn, Redding, CT (US)

(73) Assignee: 3K Anesthesia Innovations, LLP, Redding, CT (US)

(21) Appl. No.: 13/150,552

(22) Filed: Jun. 1, 2011

(51) Int. Cl. A61M 39/08

(52) U.S. Cl. 604/528

(57) ABSTRACT

An integrated oral gastric tube guide for guiding an oral gastric tube proximal to a stomach of a patient, includes a guide channel for receiving the oral gastric tube, a service channel for housing a sensor for monitoring a physiological function of the patient, a bite block for preventing a patient from occluding the guide channel; and a guide length from a top face of the bite block to a distal end of the first guide channel that comprises a length from the mouth of a patient to proximal to a stomach.

### BACKGROUND OF THE INVENTION

1. Field of the Invention

The invention relates to a medical device, specifically, an integrated oral gastric tube guide that guides a suction tube from the mouth of the patient to proximal to the stomach of the patient and that incorporates a bite block and one or more sensors for monitoring the conditions of the patient.

2. Description of the Related Art

Surgical patients can reduce their risk of certain complications and even death by having an empty stomach. In an elective, non-emergency setting, the patient is instructed to refrain from eating or drinking for several hours prior to surgery to minimize the likelihood of significant gastric contents.

However, in emergency surgery, patient non-compliance, or for certain medical conditions, such as diabetes, bowel obstruction, and obesity, the patient's stomach may not be empty and the patient will present with significant stomach contents which may be aspirated into the lungs during or immediately following the surgical procedure.

To decrease the risk of aspiration, it is possible to introduce a suction tube prior to or during surgery so that an open end of the tube is placed within the stomach. A suction force is applied to draw fluids out of the stomach. Since the tube must be placed at the beginning of the surgical procedure and must be ordinarily removed at the conclusion of the surgical procedure it is highly preferable to have the tube routed through the mouth of the patient.

A suction tube that is routed through the mouth and esophagus and into the patient's stomach is known as an oral gastric tube or orogastric tube. The procedure to position the orogastric tube in the stomach requires that the distal open end be placed into the mouth and routed via the esophagus into the stomach avoiding the trachea to avoid detouring the tube into the lungs. The procedure is difficult and tedious when, as is common, a patient's pharynx is not smooth. Pharyngeal tissue tone decreases under anesthesia which also contributes to difficulty correctly placing an orogastric tube. Substantial periods of time are often required to properly position an orogastric tube.

To improve the chances of placement and ease the procedure on the anesthesiologist, the orogastric tube must be made of a soft material and its construction flexible. However, placement of the orogastric tube is impeded if the material is too soft and/or the construction too flexible.

Orogastric tubes are available in five common sizes: 10, 12, 14, 16, and 18 French (roughly 0.125 to 0.24 inches in outer diameter). Generally, for unconscious patients, bigger tubes are easier to insert, as their construction is more rigid and therefore resist curling in the oropharynx. There are several complications that may occur with esophageal tube placement. The most serious complication is inadvertent placement into the lung, which may be complicated by hemorrhage and/or inability to ventilate the lungs. This inadvertent passage of esophageal tubes into the patient's trachea may be initially unrecognized as the cough reflex is depressed due to anesthesia.

Another problem that can be encountered during general anesthesia is that the lumen of the endotracheal tube (“ETT”) and/or orogastric tube (“OGT”) becomes occluded, partially or wholly by a patient's teeth if the patient attempts to bite down during anesthesia. Occlusion of the ETT can lead to, for example, hypoxia, hypercarbia, and the syndrome known as negative pressure pulmonary edema.

Since multiple catheter tubes, such as the endotracheal tube and/or orogastric tube are placed within the patient it is important to secure the tubes to prevent them from moving inadvertently. If an ETT moves further into the trachea it can cause lung collapse and hypoxia. If it is pulled out from the patient's mouth it can cause unintended extubation of the trachea which can also lead to hypoxia and increases the risks of pulmonary aspiration. Secure placement of an endotracheal tube is essential in patients presenting for surgery requiring one lung ventilation (OLV) using bronchial blockers. Bronchial blockers require meticulous placement within the bronchial tree so that only the parts of the lung are ventilated and other parts are not. Therefore when these tubes are in position it is extremely beneficial to have them firmly secured. It is often difficult to properly secure tubes to a patient's face to prevent movement as tapes and adhesives may be ineffective, because of the presence of facial hair, blood, perspiration, excessive soft tissue or facial trauma.

Patients having surgery have their temperature monitored continuously during the procedure. Esophageal temperature probes offer accurate, continuous measurement of core body temperature in anesthetized patients. The measured temperature can vary greatly depending on the position of the sensor in the esophagus. In the proximal esophagus, temperature is influenced by ambient air. During hypothermia, temperature in different portions of the esophagus may differ by up to 6° C. Because of the proximity of the distal esophagus to the great vessels and heart, the patient's temperature, measured from the distal esophagus, approx 30 cm from the teeth line, is most accurate and reflects changes in core temperature more quickly.

Patients receiving general anesthesia have their adequacy of ventilation continually evaluated. Qualitative clinical signs such as auscultation of breath sounds are useful. In surgical procedures performed on patients under general anesthesia, an esophageal stethoscope is usually introduced into the esophagus in order to enable monitoring of the patient's heart and breath sounds. Standard esophageal stethoscopes may not provide optimal auscultation of heart and breath sounds due to the unpredictable location within the esophagus or stomach.

Patients receiving general anesthesia are monitored with an esophageal electrocardiogram (E-ECG) to determine intraoperative myocardial ischemia. Compared with the surface electrocardiogram (ECG), the E-ECG detects significantly more ischemic episodes in both, an animal comparison study, as well as in coronary artery bypass graft surgery. The surface ECG was the best method to detect intraoperative myocardial ischemia in routine clinical practice. A recent study proves that the E-ECG represents a more sensitive method to detect intraoperative myocardial ischemia than standard 12 channel surface ECG especially in open heart surgery.

Patients receiving general anesthesia are monitored by pulse oximetry to give readings of blood oxygen saturation for many clinical purposes. However, there are significant limitations on the accuracy and the availability of pulse oximetry data in some circumstances. Pulse oximetry is a pulse-dependent technique, and any significant reduction in the amplitude of the pulsatile component of the photoplethysmographic signal can lead to dubious values for blood oxygen saturation (SpO2) or complete failure. Hence, pulse oximeters require adequate peripheral perfusion to operate accurately. When peripheral perfusion is poor, as in states of hypovolemia, hypothermia or vasoconstriction, oxygenation readings become unreliable or cease.

Such clinical situations occur, for example, after prolonged operations, especially hypothermic cardiopulmonary bypass surgery, vascular, reconstructive, neurosurgery and in patients with extensive burns. The problem arises because conventional sensors must be attached to the most peripheral parts of the body where pulsatile flow is most easily compromised. Measurements at sites other than the finger or ear, such as the forehead and nose, give no improvement in poorly perfused patients. Thus, pulse oximeter readings are often unobtainable at just the time when they would be most valuable.

Therein, it is a general object of the present invention to provide an improved guide for guiding an orogastric tube into the esophagus and proximal to the stomach of the patient for insertion into the stomach for evacuation of stomach fluids, while preventing the patient from biting and occluding tubes that are inserted orally.

Another object of the invention is to provide an improved device to secure an orogastric tube inserted orally in place at a desired depth within the patient while also preventing an endotracheal tube from dislodgment during a surgical procedure or patient positioning.

Another object of the invention is improved placement of vital sign sensors with a degree of confidence that the placement is accurate.

A further object of the invention is the reduction of valuable anesthesia time especially the time needed for placement of an orogastric tube and/or esophageal stethoscope.

### SUMMARY OF THE INVENTION

These and other needs are met by the present invention. Therein, an integrated oral gastric tube guide for guiding an oral gastric tube into a stomach of a patient, includes a guide channel for receiving the oral gastric tube, a service channel for housing a sensor for monitoring one or more physiological functions of the patient, a bite block for preventing a patient from occluding the guide channel; and a guide length from a top face of the bite block to a distal end of the first guide channel that comprises a length from the mouth of a patient to proximal to a stomach.

Moreover, an integrated oral gastric tube guide for guiding an oral gastric tube proximal to the stomach for insertion into the stomach of a patient, the integrated oral gastric tube guide includes a lumen comprising a service channel and a guide channel for guiding the oral gastric tube and a sensor disposed in the service channel for monitoring of one or more physiological functions of the patient.

Therein, other embodiments described herein meet the objectives of the present invention.

### BRIEF DESCRIPTION OF THE DRAWINGS

In the following, the invention will be described in detail by the aid of an example with reference to the attached drawings herewith.

FIG. 1 is an elevational view of an integrated oral gastric tube guide in accordance with one or more embodiments of the present invention.

FIG. 2 is a plan view of the integrated oral gastric tube guide of FIG. 1.

FIG. 3 is an end view of a portion of the integrated oral gastric tube guide of FIG. 1.

FIG. 4a is a cross-section through a portion of the integrated oral gastric tube guide taken of FIG. 1 taken at Section A-A of FIG. 1.

FIG. 4b is a cross-section through a portion of the integrated oral gastric tube guide of FIG. 1 taken at Section B-B of FIG. 1.

FIG. 4c is a cross-section through a portion of the integrated oral gastric tube guide taken of FIG. 1 taken at Section C-C of FIG. 1.

FIG. 5 is a cross-section through the bite block of integrated oral gastric tube guide of FIG. 1 taken at Section D-D in FIG. 2.

FIG. 6 is a schematic diagram of a layout of one or more sensors and the esophageal stethoscope in the service channel of the integrated oral gastric tube guide of FIG. 1.

### DETAILED DESCRIPTION OF THE INVENTION

Reference will now be made in detail to several views of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, left, right, up, down, over, above, below, beneath, rear, and front may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

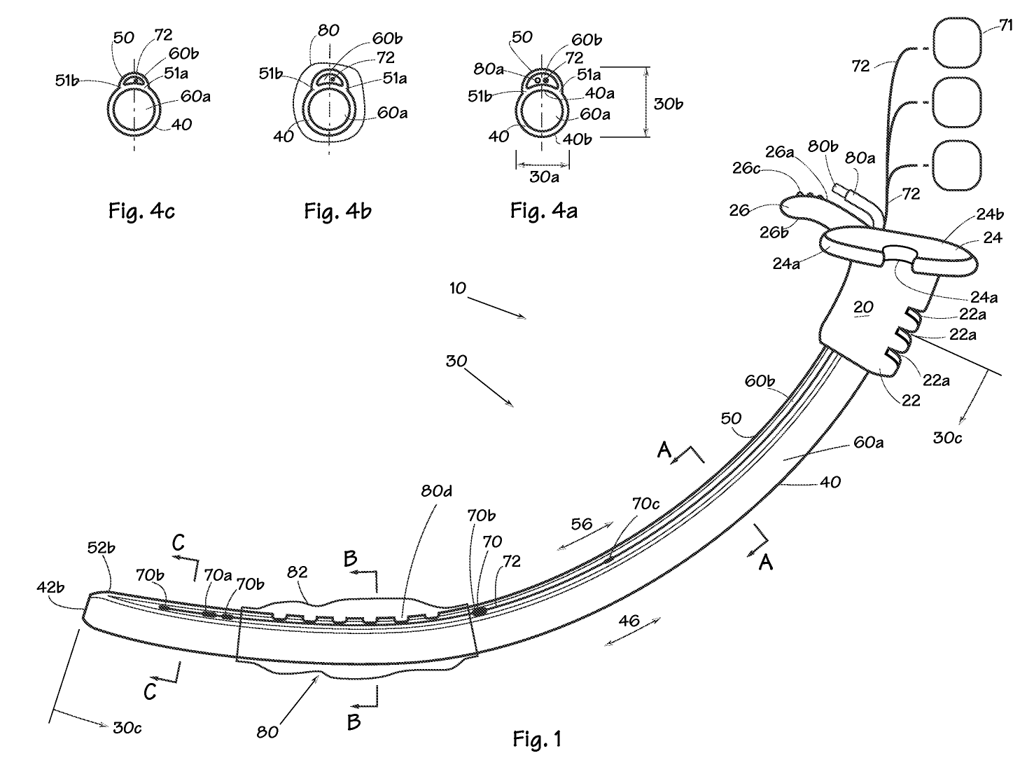

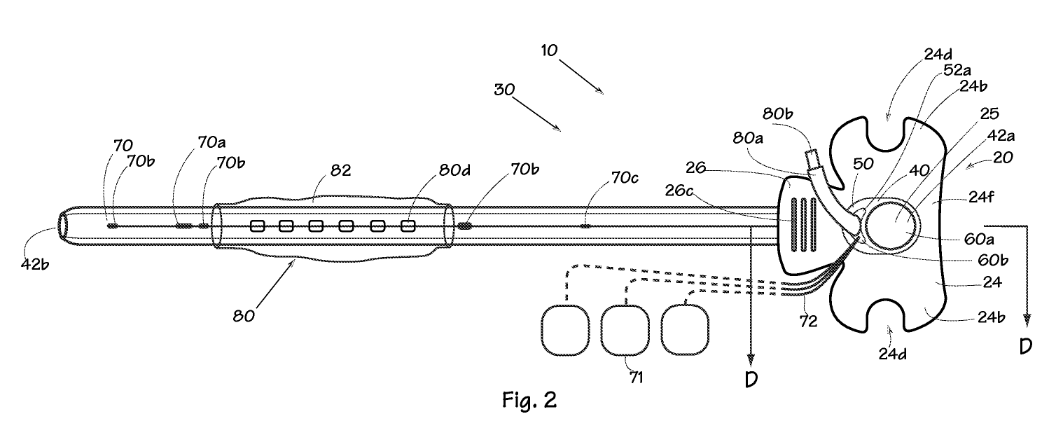

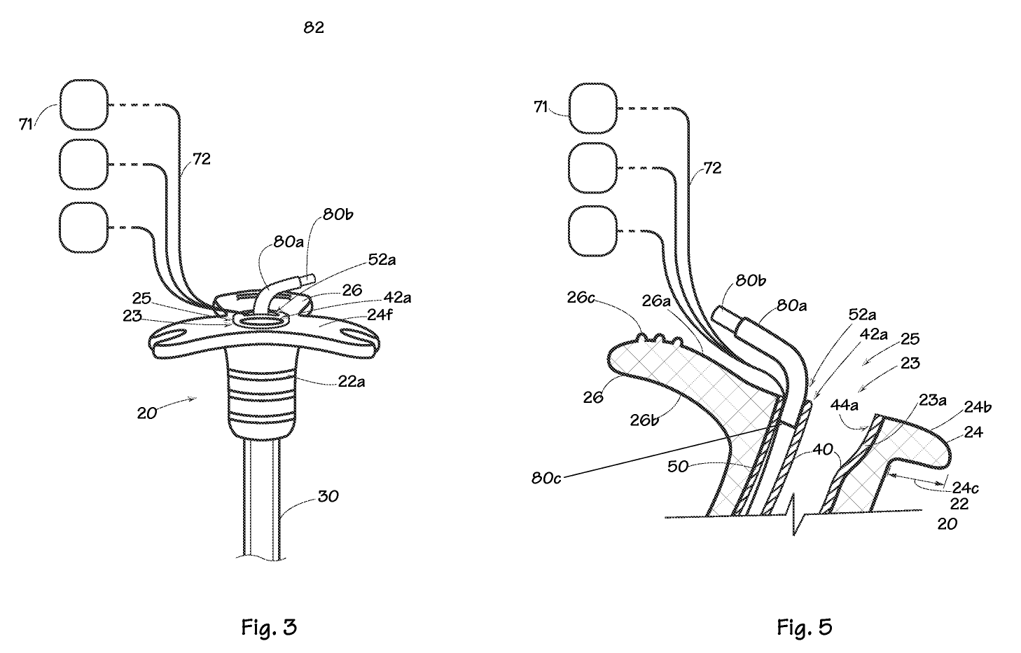

FIG. 1 is an elevational view of an integrated oral gastric tube guide in accordance with one or more embodiments of the present invention. FIG. 2 is a plan view of the integrated oral gastric tube guide of FIG. 1. FIG. 3 is an end view of a portion of the integrated oral gastric tube guide of FIG. 1. FIG. 4a is a cross-section through a portion of the integrated oral gastric tube guide taken of FIG. 1 taken at Section A-A of FIG. 1. FIG. 4b is a cross-section through a portion of the integrated oral gastric tube guide of FIG. 1 taken at Section B-B of FIG. 1. FIG. 4c is a cross-section through a portion of the integrated oral gastric tube guide taken of FIG. 1 taken at Section C-C of FIG. 1. FIG. 5 is a cross-section through the bite block of integrated oral gastric tube guide of FIG. 1 taken at Section D-D in FIG. 2.

Therein, an integrated oral gastric tube guide (“IGTG”) 10 is usable in one or more medical procedures on a patient to guide an oral gastric tube, i.e., orogastric tube, monitor one or more vital physiological functions of the patient during the one or more medical procedures, and provide a support for other medical devices needed during the one or more medical procedures.

Therein, IGTG 10 guides the orogastric tube (not shown) from a patient's mouth to proximal to that patient's stomach so that the orogastric tube can be inserted in the patient's stomach to suction gastric contents from the stomach.

The integrated oral gastric tube guide comprises
a bite block 20 for positioning the IGTG in the mouth of the patient, securing it to the face of the patient, preventing the patient from occluding the guide channel, and/or provide a support for other medical devices needed during the one or more medical procedures;
a lumen 30 comprising a first wall 40 defines at least in part guide channel 60a for receiving and guides the orogastric tube and a second wall 50 that in combination with wall 40 defines at least in part one or more service channels 60b for housing preferably one or more sensors 70 and one or more leads 72 to connect sensors 70 operatively to monitoring equipment and an esophageal stethoscope 80.

Bite block 20 is made from a resilient, sterilizable material that can withstand the forces of a human bite. Therein, bite block 20 is preferably made by injection molding and may comprise one or more thermoelastic or plastic materials.

Bite block 20 comprises a shank 22, a top portion 24, and a handle 26. Shank portion 22 is preferably substantially circular in cross-section relative to a main axis of the integrated oral gastric guide tube and is preferably substantially oval or cylindrical in shape but having a curve to accommodate the physiology of the human mouth.

Shank portion 22 comprises one or more teeth receiving portions 22a that are indented in the shank to receive the upper incisors of the patient. When multiple teeth receiving portions 22a are provided they are spaced apart from each other to provide a plurality of positions to accommodate different physiological dimensions of the human mouth. Therein, the upper incisor teeth of a patient may rest, grab, and/or engage the bite block 20 via the teeth receiving portion 22a (if only one is provided) or via one of the teeth receiving portions 22a (when more than one is provided). One or more teeth receiving portions may also be provided for the lower incisor teeth of the patient and disposed on a side opposite teeth receiving portions 22a. Teeth receiving portion 22a may be indented 2-5 mm (3 mm) relative to an outside surface of bite block 20. When multiple teeth receiving portions 22a are provided, the teeth receiving portion disposed distal from the top surface of the bite block (i.e., top surface 24) and that engages the upper incisors defines the teeth line. As taught further herein, the teeth line defines the preferred positions of one or more sensors 70 and the length of lumen 30.

A through-channel 23 is disposed through the shank substantially parallel or coincident to a main longitudinal axis of IGTG 10 and receives an end portion of lumen 30, which includes a guide channel for the orogastric tube. To prevent the orogastric tube from being occluded, i.e., blocked by the bite of the patient, bite block 20 comprises a size and/or material that prevents occlusion based on the force of a human bite known from physiological studies. Through-channel 23 may comprise an enlarged upper end 23a.

Top portion 24 comprises one or more rounded edge portions 24a, an access opening 25, a cantilevered portion 24b that is preferably disposed preferably by a cantilever distance 24c about a top end of the shank. Cantilever distance 24c from the peripheral edge of the top end of the shank is preferably sized to permit the lips of the patient to be at least partially covered by cantilevered portion 24c in one or more locations.

Access opening 25 is in communication with through-channel 23. Access opening 25 may have any suitable cross-sectional shape or size, but preferably, access opening 25 has a cross-section and/or one or more cross-sectional dimensions that are the same or smaller than the cross-section or cross-sectional dimension of through-channel 23 in order to secure the orogastric tube without play in the bite block.

Cantilevered portion 24b is also preferably sized such that surgical tape may be placed over top portion 24 to help secure the bite block to the patient's face such that the bite block, i.e., IGTG 10, is stable enough to provide a support for other medical devices via one or more indents 24d.

Indents 24d preferably are suitably sized such that facilitate the securing of other medical devices (not shown) including medical tubing, for example, by pressing and/or snapping the medical devices into one of indents 24d. Therein, indent 24d may be formed to have an arc of preferably 10 mm, which is suitable for medical tubes. One or more indents 24d may comprise one or more end portions 24e that extend outwardly into the indented space to narrow the respective indent to aid in retaining the medical device and help prevent unintended disengagement from the indent. Top portion 24 comprises a curved surface 24f in at least a transverse direction in order to conform to the patient's physiology. Top portion 24 may have any suitable size and/or shape; however, it have a planar width, i.e., a dimension perpendicular to the longitudinal axis of lumen 30 in the view of FIG. 2, of 35-45 mm (preferably 40 mm) and a length, a dimension parallel to the longitudinal axis of lumen 30 in the view of FIG. 2, of 30-40 mm (preferably 35 mm).

Handle 26 is connected to top portion 24 and is disposed to preferably extend upward from top portion 24. Handle 26 preferably comprises a curved shape in a cross-section such that the handle may be grasped and/or adjusted with a thumb placed on a top surface 26a of the handle and a portion of the index finger on a bottom surface 26b. Handle 26 may have any suitable size, but has a cross-wide width, i.e., a dimension perpendicular to the longitudinal axis of lumen 30 in the view of FIG. 2, of 15-25 mm (preferably 20 mm) and a length, i.e., a dimension parallel to the longitudinal axis of lumen 30 in the view of FIG. 2, of 10-20 mm (preferably 15 mm). One or more raised structures 26c are provided on at least top surface 26a to permit easier grasping and/or adjustment of the handle, especially by medical personnel wearing surgical gloves.

Lumen 30 comprises a first wall 40 that preferably defines at least partially guide channel 60a for receiving the orogastric tube and a second wall 50 that defines at least partially service channel 60b. Lumen 30 comprises a cross-sectional width 30a and a cross-sectional height 30b. At the distal end of bite block 20, lumen 30 may have a maximum outside cross-sectional dimensions of width 30a of 10-12 mm (preferably 11 mm) and height 30b of 12-18 mm (preferably 15 mm).

Wall 40 preferably comprises an open proximal end portion 42a preferably disposed at least partially against a bottom surface of top portion 24 and/or disposed so that a surface edge of end portion 42a is exposed and is substantially level with a top surface of portion 24. Preferably, proximal end 42a comprises an enlarged portion 44a that fits tightly within enlarged upper end 23a and permits easier insertion of the orogastric tube. Enlarged portion 44a may be made by fitting wall 40 into through-channel 23 until it abuts against the bottom surface of top portion 24 and then applying a heat treatment that enlarges the wall 40 to form enlarged portion 44a. Enlarged upper end 23a may also be formed in the same operation.

Wall 40 preferably comprises an open distal end portion 42b that is narrowed by reducing the thickness of wall 40 and lacking any sharp edges for patient safety.

Wall 40 is preferably made from polyvinyl chloride that is transparent to the naked eye for ease of operation. One or more radio opaque markers or stripes may be embedded in walls 40 in order to make IGTG 10 visible in X ray imaging or other types of medical imaging.

Wall 40 preferably comprises a curve 46 that is suitable for the physiology of the human oropharynx and/or esophagus, and therein may have a degree of curvature of 5-15 degrees over the guide length of lumen 30. Wall 40 preferably has a circular cross-section, although other cross-sectional shapes may also be used.

Preferably, wall 40 may have a uniform wall thickness but may have also have enlarged wall thickness and/or stabilizing structures where necessary to maintain rigidity and structural integrity. Therein, wall 40 may have a wall thickness of 0.5 mm to 2.0 mm; a wall thickness of 0.5 mm to 2.0 mm may be used when the wall thickness is constant between proximal end portion 42a and distal end portion 42b. However, the wall thickness may be non-constant wherein a thickness of 0.5 mm to 1.5 mm is used towards the end portions and a middle portion may have a thickness of 1.0 mm to 2.0 mm, respectively.

The orogastric tube is received in guide channel 60a and is guided from the patient's mouth to proximal to the patient's stomach so that the orogastric tube can be inserted in the patient's stomach to suction gastric contents from the stomach. Therein, guide channel 60a is defined by wall 40, but in accordance with one or more embodiments of the present invention wall 40 may not be present in the bite block and guide channel 60a may be defined by partitioning through-channel 23 into a through-channel solely dedicated for guide channel 60a and/or guide channel 60a is defined enlarged upper end 23a.

Guide channel 60a may be sized to have a nominal diameter of 9 French to 34 French (also known as Charriere gauge, i.e., 3.0 mm to 11.3 mm except in or near end portions 42a and 42b and/or enlarged upper end 23a. Preferably, guide channel has a dimension of 8 mm diameter.

Wall 50 in combination with wall 40 defines at least partially service channel 60b. Wall 50 preferably comprises an open proximal end portion 52a disposed at least partially against a bottom surface of top portion 24 and/or disposed so that a surface edge of end portion 52a is exposed and is substantially level with a top surface of portion 24. Wall 50 further comprises a closed distal end portion 52b.

Wall 50 is preferably made from polyvinyl chloride that is transparent to the naked eye for ease of operation. One or more radio opaque markers or stripes may be embedded in walls 50 in order to make IGTG 10 visible in X ray imaging or other types of medical imaging.

Wall 50 preferably comprises a curve 56 that matches curve 46 that is suitable for the physiology of the human oropharynx and/or esophagus, and therein may have a degree of curvature of 5-15 degrees over the guide length of lumen 30. Wall 50 preferably has arcuate cross-section that joins onto an outer wall portion of wall 40 at wall junctures 51a and 51b, although other cross-sectional shapes may also be used.

Preferably, wall 50 may have a uniform wall thickness but may have also have enlarged wall thickness where necessary to maintain rigidity and structure. Therein, wall 50 may have a wall thickness of 0.3 mm to 2.0 mm; a wall thickness of 0.3 mm to 2.0 mm may be used when the wall thickness is constant between proximal end portion 52a and distal end portion 52b. However, the wall thickness may be non-constant wherein a thickness of 0.3 mm to 1.5 mm is used towards the end portions and a middle portion may have a thickness of 1.0 mm to 2.0 mm, respectively.

Therein, the ratio of the thickness of wall 50 to wall 40 preferably may be in the range of 0.3 to 1.5, wherein a ratio range of 0.35-0.7 is preferred since it keeps the lumen most flexible and yet prevent unintended curling for channels 60a that are sized 12 French to 24 French. For example, wall 50 may have a wall thickness of 0.70 mm and wall 40 may have a wall thickness of 1.0 mm. In this manner, lumen 30 is kept flexible and has the least amount of cross-sectional size.

In accordance with one or more embodiments of the present invention, a portion 40a of wall 40 connecting junctures 51a and 51b via the shortest route in cross-section has a wall thickness may have a different wall thickness than a portion 40b of wall 40 connecting junctures 51a and 51b via the longest route in cross-section. When wall 40a is thicker than wall 40b and/or wall 50 permits wall 40a to be used as a stiffener and therein walls 50 and/or walls 40b to have thin wall section and yet avoid lumen 30 to avoid curling.

One or more sensors 70 are disposed in service channel 60b. Sensors 70 may include one or more of the following:
an esophageal temperature sensor 70a,
one or more esophageal electrocardiogram sensors 70b,
a pulse oximeter sensor 70c, and/or
any other sensor 70 able to monitor one or more physiological conditions of the patient.

Therein, preferably lumen 30, but at least wall 50 is preferably heat transmissible so that the esophageal temperature sensor may be operatively disposed in channel 60b, adapted to permit the esophageal electrocardiogram sensor to be operatively disposed in channel 60b or outside of channel 60b, light transmissive in order to permit the pulse oximeter sensor 70c to be operatively disposed in channel 60b if it is a direct-absorption pulse oximeter sensor and/or infrared light passive if the pulse oximeter sensor 70c is based on measuring the infrared light absorption of oxygenated and deoxygenated hemoglobin, and sound transmissible to permit the esophageal stethoscope sensor to be operatively disposed in channel 60b or a combination of one or more sensors are disposed therein.

Each sensor 70 is connected by separate lead 72 that is disposed in channel 60b and exits through proximate end portion 52a or via a port provided in the bite block. Each lead is preferably operative with one or more monitoring devices. To place sensors 70 in operative condition one or more leads may terminate in a plug 71 useful for connecting to the respective monitoring device or devices. One or more sensors 70 may share one or more leads 72 using technology known in the art to share multiple data streams on one lead. One or more leads may terminate into a unitary terminal plug so that minimal number separate plugs are provided. Sensors 70 may also be arranged in a bus and connected operatively to monitoring equipment (not shown). However, in accordance with one or more embodiments of the present invention, the esophageal stethoscope 80 may comprise a transducer and also have lead as described below.

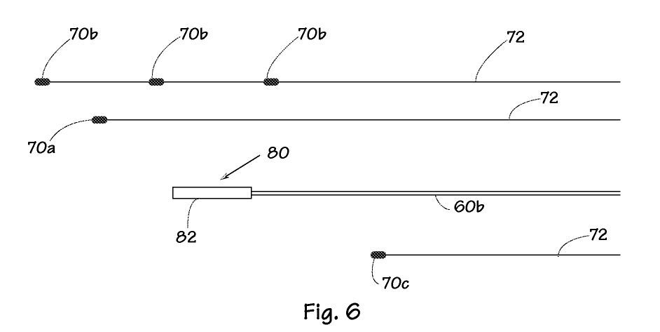

FIG. 6 is a schematic diagram of a layout of one or more sensors and the esophageal stethoscope in the service channel of the integrated oral gastric tube guide of FIG. 1. Preferably sensors 70 are disposed at predetermined positions. Therein, as noted above, lumen 30 preferably comprises a guide length 30c from the teeth receiving portion 22a when one teeth receiving portion is present (or from the center-most, i.e., middle, teeth receiving portion when more than one teeth receiving portion is present) to a distal end of the first guide channel that comprises a length of approximately 35 cm.

In the known art, esophageal temperature is usually measured with an electric flexible temperature probe and the esophageal temperature probe is a flexible tube and is easily malpositioned within the patient. Such probes may become lodged in an abutment against the pharynx and begin to double up and fold into the mouth. Such probes may also enter tracheobronchial tree, perforate esophageal wall and even be found in the pleural space.

Esophageal temperature sensor 70a overcomes these problems since it is integral with the IGTG 10. Preferably, an esophageal temperature sensor 70a is disposed near distal end 52b of the service channel. Esophageal temperature sensor 70a is preferably located 31 cm from the teeth line. Thus, esophageal temperature sensor 70a is securely located at the patient's distal esophagus with a high degree of confidence. Even more advantageously, IGTG 10 achieves locating the esophageal temperature sensor 70a without trauma to the patient's pharynx.

Esophageal cardiogram sensors 70b improve on the known art by being located in a position wherein a lower electrical impedance, a better electrical conductivity, and the close anatomic range between the heart and the electrode are possible with a higher degree of confidence than the same are possible with a conventional esophageal electrocardiogram. Specifically, esophageal cardiogram sensors 70b are a plurality (preferably three electrodes) disposed spaced-apart n channel 60b, i.e., under a wall) or directly embedded in wall 50 so that a top portion of the electrode is in direct contact with the patient or placed adjacent outside of the wall so that the electrode is in direct contact with the patient.

By being placed in the lower esophagus, in the area close to the left atrium; a characteristic biphasic “P” wave can be obtained from esophageal cardiogram sensors 70b when the data is analyzed. The first esophageal cardiogram sensor 70b is preferably disposed approximately 32 cm from the teeth line, the second sensor is preferably spaced 2.5 cm away from the first, i.e., approximately 29.5 cm from the teeth line, the third is spaced the same distance from the second, i.e., approximately 27 cm from the teeth line. The distances from the teeth line may be adjusted to avoid interference with sound chamber 82, described below.

Therein, the signals of esophageal cardiogram sensors 70b can be detected unfiltered and free of artifacts. In a conventional esophageal electrocardiogram, it may not be possible to place electrodes optimally because of involvement in the surgical field, electrodes may be lost during the surgical procedure, and spatial limitations may limit the ability of the anesthesia care team to replace electrodes.

Pulse oximeter sensor 70c provides more reliable oxygen saturation readings than surface pulse oximetry in patients, especially in situations of hemodynamic instability. Pulse oximeter sensor 70c is preferably disposed approximately 19 cm from the teeth line. The distance from the teeth line may be adjusted to avoid interference with sound chamber 82, described below.

Esophageal stethoscope 80 transmits the mechanical sound waves of a sound chamber 82 that extends 360 degree around the lumen via service channel 60b to a monitoring line 80a. Line 80a has a terminal end 80b for connecting to physician's stethoscope lead or into an electromechanical monitor and a distal open end 80c that is held in place, by press-fit, gluing, hot melt, and/or any other suitable means, near the proximate end of the service channel. A plurality of openings 80d is disposed in wall 50 to permit air movement along the service channel to end 80c and a stethoscope or monitoring device connected to terminal end 80b. Therein, the mechanical sound waves of sound chamber 82 travel through openings 80d and from there through service channel 60b to monitoring line 80a. From there the sound waves travel to end 80b and therefrom to the physician's stethoscope lead or into the electromechanical monitor. The sound chamber preferably comprises a first end portion approximately 20 cm from the teeth line and a second end portion approximately 27 cm from the teeth line.

However, esophageal stethoscope 80 may in addition or instead comprise a transducer that transforms the mechanical waves to electrical impulses. Therein, a sound chamber is not necessary and the transducer is connected via a lead 72 to a monitoring unit, as may be known in the art.

Herein, IGTG 10 conforms to the standards for intraoperative monitoring that have been adopted by the American Society of Anesthesiologists (ASA). As noted previously, these standards apply to all general anesthetics, regional anesthetics, and monitored care. According to these standards “every patient receiving anesthesia shall have temperature monitored when clinically significant changes in body temperature are intended, anticipated or suspected”. The temperature of patients undergoing general anesthesia (GA) should be monitored always. Intraoperatively, temperature is usually measured by a thermistor or thermocouple. Disposable thermistor or thermocouple probes are available for monitoring the temperature of the tympanic membrane, rectum, nasopharynx, esophagus, bladder, and skin. Esophageal temperature sensors, often incorporated into esophageal stethoscopes, provide the best combination of economy, performance, and safety.

IGTG 10 comprising an esophageal temperature sensor 70a has several advantages over existing and widely used esophageal probes. For example, as discussed further herein, because IGTG 10 includes a plurality of teeth receiving portions 22a, IGTG 10 is stabilized at the patient's incisors and, thus, esophageal temperature sensor 70a preferably is properly located in the most advantageous position within the esophagus.

Advantageously, IGTG 10 saves valuable operating room (“OR”) time by simplifying the placement of one or more sensors, is easy to store, is easy to use, is cost-effective, simplifies the combined placement of the esophageal probe and bite block, improves performance of the esophageal probe by its more accurate placement, provides more reliable temperature information, and provides a more stable placement in a patient's mouth for the endotracheal tube and orogastric tube.

In a conventional procedure, an anesthesiologist or other qualified user intubates an anesthetized patient's trachea with an endotracheal tube. The orogastric tube is then inserted to the side of the endotracheal tube. An esophageal temperature probe is inserted and located. Since the OGT is a soft and flexible tube, positioning a OGT can be a difficult and tedious task and sometimes requires substantial periods of time. Placement of the esophageal temperature probe is a separate procedure requiring additional time to properly position within the esophagus. To prevent the anesthetized patient's teeth from closing and/or obstructing the ETT and OGT, a bite block is inserted to keep the upper and lower jaw separated.

In use, a patient is anesthetized and intubated. IGTG 10 is removed from its packaging. IGTG 10 is provided in a sealed and sterilized packaging for single-use application and provided in proper sizes, for one or more sizes for pediatric patients (including youth patients) and one or more sizes for adult patients.

Since IGTG 10 is an integrated device, the guide for an OGT, one or more sensors, and a bite block are provided in one unit whereas these are conventionally provided separately, and the correct sizing provides the proper size for the patient based on standard anatomical sizing. In this way, by aligning the patient's upper incisors to one of the teeth receiving portions 22a, the IGTG is properly located. That is, the one or more sensors and distal end are properly located for maximum efficacy.

Therein, IGTG 10 is placed in the anesthetized patient's mouth. The IGTG is correctly located in the esophagus by aligning the patient's upper teeth in the teeth receiving portion 22a if only one is provided or in a preferred teeth receiving portion 22a if multiple teeth receiving portions are provided. This locates the distal end of the IGTG near the stomach and locates the one or more sensors properly. The anesthesiologist then inserts the orogastric tube in guide channel 60a. The removal is the reverse of the placement. Thus, placing the OGT using IGTG takes less time than locating the OGT alone.

Herein, IGTG 10 easy to storage and easy to work with since it is a single device and can be packaged together with an OGT.

IGTG 10 can be manufactured for less than existing esophageal probe and bite block together. According to the American Society of Anesthesiologists “every patient receiving general anesthesia shall have the adequacy of ventilation continually evaluated. Qualitative clinical signs such as chest excursion, observation of the reservoir breathing bag, and auscultation of breath sounds are useful”. In other words auscultation is optional, not a mandatory monitor for the anesthetized patient. Auscultation of breath and heart sounds can be performed by an esophageal or precordial stethoscope. The quality of breath and heart sounds is much better with an esophageal stethoscope. IGTG 10, because of the accuracy of its placement, can further optimize heart and breath sounds than a standard esophageal stethoscope. Therein, because

IGTG 10 includes a plurality of teeth receiving portions 22a, IGTG 10 is stabilized at the patient's incisors and, thus, sound chamber 80 preferably positions directly behind the patient's heart for optimally determining the heart and breath sounds of the patient.

IGTG 10 increases safety of the esophageal tube placement. Rarely, the stethoscope slides into the trachea instead of the esophagus, resulting in a gas leak around the endotracheal tube cuff. Inadvertent placement of an OGT into the lung may be complicated by pneumothorax, hemorrhage and inability to ventilate the lungs. Because the cough reflex is depressed due to anesthesia, passage of the OGT or stethoscope into the tracheobronchial tree is initially unrecognized.

Advantageously, IGTG 10 reduces incidence of the tube misplacement. Generally, for anesthetized patients, bigger tubes are easier to insert, as they are more rigid and therefore resist curling in the oropharynx.

IGTG 10 reduces a patent's postoperative throat discomfort. Placement through the mouth can occasionally cause mucosal irritation and bleeding. Intraoral bleeding occurs routinely if OGT requires multiple attempts to place it.

Because of the bigger tube diameter and the softer tip, IGTG will protect the oral mucosa from excessive irritation and will reduce the incidence of the intraoral bleeding and sore throat.

IGTG 10 provides more reliable temperature information. To avoid measuring the temperature of ambient air within the esophagus, the temperature sensor should be positioned behind the heart in the lower third of the esophagus. Existing temperature probes can be easily misplaced into the esophagus or even into the stomach of the patients.

IGTG 10 was designed with optimal distance between the bite block proximally and the temperature monitor distally in the adult. Bite block placement at the level of the upper teeth will be always associated with the optimal temperature sensor placement in the lower third of the esophagus.

IGTG 10 provides a more stable placement in a patient's mouth than conventional endotracheal tube/orogastric tube placement. Bite block 22 prevents IGTG 10 and the ETT and/or OGT from being inadvertently advanced into the patient or retracted from the patient's mouth. Securing an ETT to minimize any movement within the patient's oropharynx is essential in patients presenting for surgery requiring one lung ventilation (OLV) using bronchial blockers because the blocker needs to be maintained in a very specific position within the patient's bronchial tree to properly ventilate the lungs.

IGTG 10 prevents lumen 30 from being occluded by a patient's teeth. Bite block 22 prevents the guide channels from being occluded by a patient's teeth. The bite block is effective in keeping a patient's jaw open and thus preventing the teeth from clamping down on the IGTG, ETT and/or OGT. Occlusion of the ETT can lead to, hypoxia, hypercarbia, and the syndrome known as negative pressure pulmonary edema.

In ITGT 10, advantageously, the information provided by an esophageal stethoscope may include confirmation of ventilation, quality of breath sounds (for example, wheezing), regularity of heart rate, and quality of heart tones. Quality of breath and heart sounds is much better with an esophageal stethoscope and many anesthesiologists believe that intubated patients should always be monitored with this device. Therein esophageal stethoscope 80 can be incorporated into IGTG 10 and precisely positioned in the area of the esophagus adjacent to the mediastinum. IGTG 10 with esophageal stethoscope 80 can be particularly useful in patients with preexisting lung disease (for example, COPD, asthma or bleomycin toxicity).

Pulse oximeters are mandatory intraoperative monitors. In addition to oxygen saturation, pulse oximeters provide an indication of tissue perfusion (pulse amplitude) and measure heart rate. Pulse oximeters have some severe limitations. The vascular bed to be monitored must be pulsatile. When peripheral perfusion is poor, as in states of hypovolemia, hypothermia, vasoconstriction, low cardiac output and low mean arterial pressure, oxygenation readings become extremely unreliable.

Conventional transmission sensors are usually placed on the most peripheral parts of the body such as the finger, the ear lobe or the toe, where pulsatile flow is most easily compromised. Pulse oximeters can fail in patients undergoing prolonged procedures such as, cardiac, vascular or neuro-surgery due to cooling and poor peripheral perfusion. IGTG 10 with the pulse oximeter sensor 70c may be able to monitor the photoplethysmographic (PPG) signals from the patient's esophagus and can be very useful in patients with compromised peripheral circulations.

IGTG 10 having a plurality of sensors 70, such as temperature sensor 70a, electrocardiogram sensor 70b, and pulse oximeter sensor 70c, preferably combines three essential monitors in one simple and easy to use device. The main indication to use this device is any emergency situations where early and reliable monitoring is essential in diagnosing and treating live threatening conditions. This device can be useful not only in the hospital, but outside the hospital as well.

The simplicity and reliability of this device allows practitioners to use it on the field and during transport patient to the medical facility.

Another indication for use of IGTG 10 having a plurality of sensors 70, such as temperature sensor 70a, electrocardiogram sensor 70b, and pulse oximeter sensor 70c, is in any patient in the pre-hospital setting, including trauma patients. IGTG 10 having a plurality of sensors 70, such as temperature sensor 70a, electrocardiogram sensor 70b, and pulse oximeter sensor 70c, can be placed easily by paramedics after securing the patient's airway with an endotracheal tube.

Compare with currently used monitors, IGTG 10 has a several advantages:
a. Less time to place and obtain the first monitor data.
b. All monitors in one device, easy to carry
c. More reliable readings, less wires.

Any intubated patient that is being transported from one medical facility to another facility will also benefit from ITGT 10 by:
a. More reliable monitor readings, less wires, easier to move patients
b. Provides improved security and stability of tracheal tube with respect to a patient's mouth.

IGTG 10 is illustrated herewith and dimensions are provided for a size suitable for an adult patient. Therein, IGTG 10 is preferably provided in proper sizes, for one or more sizes for pediatric patients (including youth patients) and one or more sizes for adult patients in the sizes given in Table 1.

TABLE 1
Sizes of IGTG 10

| | | Maximum | | |
|---|---|---|---|---|
| | | Cross- | Maximum Cross- | Guide |
| | Comparable | sectional Width | sectional Height | Length |
| Age of | Size | 30a in mm; | 30b in mm, | 30c in cm, |
| Patient | of OGT in | (preferred in | (preferred in | (preferred in |
| (Years) | French | parenthesis) | parenthesis) | parenthesis) |
| 4 ± 2 | 10 | 7-9 (8) | 9-15 (12) | 19-21 (20) |
| 8 ± 2 | 12 | 8-10 (9) | 10-16 (13) | 23-25 (24) |
| 12 ± 2 | 14 | 9-11 (10) | 11-17 (14) | 27-29 (28) |
| 16 ± 2 | 16 | 10-12 (11) | 12-18 (15) | 31-33 (32) |
| 18 and | 16-18 | 10-12 (11) | 12-18 (15) | 34-36 (35) |
| older | | | | |

In accordance with one or more embodiments of the present invention, IGTG 10 may also be manufactured by molding, preferably injection molding, lumen 30 and/or bite block 20 by itself and/or in combination with each other and then placing by glue and/or any other suitable means one or more sensors 70 and one or more leads 72 in service channel 60b. Line 80a is preferably made separately and is then press-fit into the lumen while the lumen is still hot so that when the lumen cools a tight fit is achieved. Alternatively, line 80a may be glued or held in place in any other suitable means. Sound chamber 82 is preferably made separately and then is glued, sonically welded, and/or fitted to a finished lumen 30 by any suitable means.

In accordance with one or more embodiments of the present invention, IGTG 10 may be manufactured by providing sensors 70 and leads 72, which are operatively connected to each other. One or more sensors 70 are then disposed in predetermined positions in cavity formed made in one or more mold of a mold apparatus, preferably an injection molding machine. Therein, the one or more mold parts define the lumen. If necessary, the one or more sensors 70 are spaced from the surface of the mold's cavity using spacers, i.e., blocking, known in the art. One or more leads 72 are suitably arranged and also spaced from the surface of the mold's cavity using spacers, i.e., blocking, known in the art.

Lumen 30 is molded using any suitable molding apparatus, including using camming to obtain guide channel 60a and/or service channel 60b. Therein, the one or more sensors 70 and/or the one or more leads 72 are embedded preferably in wall 50. Leads 72 have are suitably long enough to extend beyond the end of lumen 30.

Therein, preferably, the one or more sensors 70 and/or the one or more leads 72 are suitably selected to withstand the pressure and/or heat from the molding process. Thus, in the present embodiment rather having the sensors disposed in the service channel, the one or more sensors 70 and/or one or more leads 72 may be embedded in the wall of the lumen.

The mold parts may be changed and/or left in place to mold bite block 20 over and/or around the proximal end of the lumen using injection molding or any other suitable molding technique. Line 80a is preferably made separately and is then press-fit into the lumen while the lumen is still hot so that when the lumen cools a tight fit is achieved. Alternatively, line 80a may be glued or held in place in any other suitable means. Sound chamber 82 is preferably made separately and then is glued, sonically welded, and/or fitted to a finished lumen 30 by any suitable means.

While the invention has been described in conjunction with specific embodiments, it is to be understood that many alternatives, modifications, and variations will be apparent to those skilled in the art in light of the foregoing description.

What is claimed is:

1. An integrated oral gastric tube guide for guiding an oral gastric tube proximal to a stomach of a patient, the integrated oral gastric tube guide comprising:
a guide channel for receiving the oral gastric tube;
a service channel for housing a sensor for monitoring a physiological function of the patient; and
a guide length from a top face of the bite block to a distal end of the first guide channel that comprises a length from the mouth of a patient to proximal to a stomach.

2. The integrated oral gastric tube guide of claim 1, further comprising a bite block for preventing a patient from occluding the guide channel.

3. The integrated oral gastric tube guide of claim 1, wherein the sensor comprises one of an esophageal temperature sensor, an esophageal cardiogram sensor, or a pulse oximeter sensor

4. The integrated oral gastric tube guide of claim 1, wherein the sensor comprises two of an esophageal temperature sensor, an esophageal cardiogram sensor, or a pulse oximeter sensor.

5. The integrated oral gastric tube guide of claim 1, wherein the sensor comprises an esophageal temperature sensor, an esophageal cardiogram sensor, and a pulse oximeter sensor.

6. The integrated oral gastric tube guide of claim 1, further comprising an esophageal stethoscope.

7. The integrated oral gastric tube guide of claim 3, further comprising an esophageal stethoscope.

8. The integrated oral gastric tube guide of claim 6, further comprising a sound chamber, and wherein the esophageal stethoscope comprises a monitoring line.

9. The integrated oral gastric tube guide of claim 7, further comprising a sound chamber, and wherein the esophageal stethoscope sensor comprises a monitoring line.

10. The integrated oral gastric tube guide of claim 1, wherein the service channel comprises a closed end.

11. The integrated oral gastric tube guide of claim 1, further comprising a first wall defining the guide channel and a second wall defining the service channel.

12. The integrated oral gastric tube guide of claim 11, wherein the first wall has a wall thickness greater than a wall thickness of the service channel.

13. The integrated oral gastric tube guide of claim 2, wherein the bite block comprises a through-channel for receiving the first wall and the second wall.

14. The integrated oral gastric tube guide of claim 2, wherein the bite block comprises a teeth receiving portion.

15. An integrated oral gastric tube guide for guiding an oral gastric tube into a stomach of a patient, the integrated oral gastric tube guide comprising:
a lumen comprising a service channel and a guide channel for guiding the oral gastric tube;
a sensor disposed in the service channel for monitoring a physiological function of the patient.

16. The integrated oral gastric tube guide of claim 15, wherein the service channel is comprises an open end and a closed end.

17. The integrated oral gastric tube guide of claim 15, wherein the lumen comprises a first wall defining the guide channel and a second wall partially defining the service channel, wherein a ration of wall thicknesses of the second wall to the first wall is 0.3 to 1.5.

18. The integrated oral gastric tube guide of claim 15, further comprising a plurality of sensors, the sensors being spaced distal from each other.

19. The integrated oral gastric tube guide of claim 18, wherein one of the sensors is an esophageal temperature sensor and is disposed near a distal end of the service channel.

20. The integrated oral gastric tube guide of claim 15, further comprising a bite block having a through-channel for receiving a portion of the lumen.
